(19) United States
(12) Patent Application Publication
(10) Pub. No.: US 2017/0112381 A1
(43) Pub. Date: Apr. 27, 2017

(54) HEART RATE SENSING USING CAMERA-BASED HANDHELD DEVICE

(71) Applicant: Xerox Corporation, NORWALK, CT (US)

(72) Inventors: Abhishek Kumar, New Delhi (IN); Kuldeep Yadav, Haryana (IN); Mridula Singh, Bijnor (IN); Himanshu J. Madhu, Mumbai (IN); Agastya Nanda, New Delhi (IN)

(21) Appl. No.: 14/921,032

(22) Filed: Oct. 23, 2015

(51) Int. Cl. A61B 5/00; G06K 9/00; A61B 5/16; G06F 3/041; H04N 5/232; A61B 5/0205; H04N 5/77; H04N 5/235

(52) U.S. Cl. CPC A61B 5/0077; H04N 5/77; G06K 9/00255; H04N 5/2351; G06F 3/0416; H04N 5/23219; A61B 5/0205; A61B 5/165; A61B 5/4884; A61B 5/02416

(57) ABSTRACT

A processor of a handheld apparatus receives orientation signals from an orientation sensor of the handheld apparatus. The orientation signals indicate the direction that a camera of the handheld apparatus is aimed. The processor can also receive touch signals from a touch sensor of the handheld apparatus. The touch signals can indicate that the handheld apparatus is contacting a user (e.g., is being held by the user). The processor controls the camera to automatically obtain video based on, for example, the orientation signals indicating that the direction that the camera is aimed is within a previously established acceptable orientation range. The processor automatically recognizes items within the video to identify a face within the video, and automatically analyzes changes of the face within the video over time to identify the heart rate, respiration rate, emotional state, stress level, etc., exhibited by the face within the video.

### BACKGROUND

Systems and methods herein generally relate to devices that determine heart rate and more particularly to heart rate sensing using a camera-based handheld device.

Major health risks such as diabetes, heart attack, stress, stroke, and cancer are on the rise. To avoid these risks, continuous monitoring of healthcare vital parameters is useful for timely detection of these diseases as well as to offer recommendations and prescriptions to the users. In the last few years, wearable devices have become available with the aim to sense physical activity, sleeping patterns, heart rate, etc. Vulnerability to a certain number of health risks can also be pointed out using the sensed data, and thus preventive or corrective measures can be suggested to the users of the system. However, wearable devices may not be used regularly, and many people stop using them after just a short time. Some known problems with wearable devices include that they are obtrusive and users often forget to use them.

Thus, non-invasive sensing techniques have also been developed. Such non-invasive sensing items can, for example, use a camera (both desktop and mobile phone) to measure heart rate. These products require users to initiate heart rate sensing by placing their fingertip on the smartphone camera to detect photoplethysmograph signals, or using facial video recordings while the user remains very still in front of a camera. These products require the users to manually start a heart rate tracking application periodically or to take action in response to periodic notifications. However, users often miss notifications and/or forget to periodically use such applications and devices in the real world.

### SUMMARY

Exemplary handheld apparatuses herein include, among other components, a processor, a camera operatively (meaning directly or indirectly) connected to the processor, an orientation sensor (which can be one or more devices such as a gyroscope, an accelerometer, etc.) operatively connected to the processor, a touch sensor and a light sensor operatively connected to the processor, etc. The processor receives, for example, orientation signals from the orientation sensor(s) indicating the direction that the camera is aimed, touch signals from the touch sensor indicating the handheld apparatus is contacting the user (e.g., is being held in the user's hand), etc.

The processor controls the camera to automatically obtain video based on, for example, the touch signals indicating the handheld apparatus is contacting the user and the orientation signals indicating the direction that the camera is aimed is within a previously established acceptable orientation range (e.g., at an angle toward the user's face). Thus, in one example, the previously established acceptable orientation range can be an angular range (e.g., relative to a gravitational source, such as the surface of the earth) having a sufficiently high likelihood of pointing from the hand of the user to the face of the user.

Additionally, the processor can receive motion signals from the accelerometer that indicate the amount of movement of the camera; and the processor can be limited to automatically obtaining video only when the amount of movement of the camera is below a previously established movement limit. Similarly, the processor can receive illumination signals from the light sensor indicating the illumination level of the environment surrounding the camera; and the processor can control the camera to automatically obtain video only when the illumination level is above a previously established illumination limit. Also, the processor can control the camera to automatically obtain video only when an application involving extended user interaction with the graphic user interface of the handheld apparatus (e.g., texting, gaming, video conferencing, etc.) is actively being used by the user of the handheld apparatus.

The processor automatically recognizes items within the video to identify the user's face within and the video. However, to conserve processing resources, the processor may be limited to automatically processing the video only when the video captures the face in a limited movement state for more than a previously established amount of time. If permitted, the processor can then automatically analyze changes of the face within the video over time to identify the heart rate, respiration rate, emotional state, stress level, etc. exhibited by the face within the video. More specifically, the processor can analyze changes of the face by identifying volumetric changes in blood vessels visible on the face (e.g., based upon fluctuations in amount of ambient light reflected from such blood vessels).

Exemplary methods herein receive, by a processor of a handheld apparatus, orientation signals from an orientation sensor of the handheld apparatus. The orientation signals indicate the direction that a camera of the handheld apparatus is aimed. Such methods can also receive, by the processor, touch signals from a touch sensor, illumination signals from a light sensor, etc. The touch signals indicate whether the handheld apparatus is contacting the user.

The processor controls the camera to automatically obtain video based on, for example, the orientation signals indicating that the direction that the camera is aimed is within a previously established acceptable orientation range and/or the touch signals indicating that the handheld apparatus is contacting the user. This previously established acceptable orientation range can be an angular range, relative to a gravitational source, having a likelihood of pointing from the hand of the user to the face of the user.

Additionally, such methods can receive motion signals from the accelerometer that indicate the amount of movement of the camera; and the methods can be limited to automatically obtaining video only when the amount of movement of the camera is below a previously established movement limit. Similarly, the methods can receive illumination signals from the light sensor indicating the illumination level of the environment surrounding the camera; and the methods can control the camera to automatically obtain video only when the illumination level is above a previously established illumination limit. Also, such methods can control the camera to automatically obtain video only when an application involving extended user interaction with the graphic user interface of the handheld apparatus is actively being used by the user of the handheld apparatus.

These methods automatically recognize items within the video to identify a face within the video; however, these methods may conserve processing resources by only analyzing changes of the face when the video captures the face in a limited movement state for more than the previously established time. If permitted, such methods automatically analyze changes of the face within the video over time to identify the heart rate, respiration rate, emotional state, stress level, etc., exhibited by the face within the video using the processor. For example, the process of analyzing changes of the face can include identifying volumetric changes in blood vessels visible on the face, based upon fluctuations in amount of ambient light reflected from the blood vessels.

These and other features are described in, or are apparent from, the following detailed description.

### BRIEF DESCRIPTION OF THE DRAWINGS

Various exemplary systems and methods are described in detail below, with reference to the attached drawing figures, in which:

FIG. 1 is a flow diagram of various methods herein;

FIG. 2 is a schematic diagram illustrating devices herein; and

FIG. 3 is a schematic diagram illustrating a user operating devices herein.

### DETAILED DESCRIPTION

As mentioned above, it is useful to periodically monitor vital signs, such as heart or respiration rates. Some known problems with wearable devices that monitor heart or respiration rates include that they are obtrusive and users often forget to wear them. Other non-wearable products require the users to manually start a heart rate tracking application periodically, or to take action in response to periodic notifications. However, users often miss notifications and forget to periodically use such applications and devices in the real world.

In view of this systems and methods herein discover opportunistic moments when the camera (e.g., front camera of a smartphone) can be switched on to measure heart rate, based on position of the phone, movement, illumination, etc., passively and without requiring user interaction. Therefore, the systems and methods herein periodically measure heart rate, respiration rate, emotional state, stress level, etc., passively without notification to the user and provide pervasive non-obtrusive sensing devices that are used in sustained adoption in real world usage situations.

While systems and methods could always keep the camera on and obtain continuous video, such would consume excess memory and processing resources, and have a negative impact on battery life. Therefore, the systems and methods herein only acquire and process video when the systems and methods herein automatically sense the right opportunity. For example, the systems and methods herein look for times when there are appropriate lighting conditions and the camera is still, and when the camera is in a position to obtain video of the user's face.

Specifically, the systems and methods herein use hard sensors already present in most handheld devices such as smartphones (e.g., accelerometer, proximity sensor, light sensor etc.) along with soft sensors (e.g., backlight on/off, application running, touch events etc.) as a proxy to discover “opportunistic moments” where the smartphone's front camera can be switched on to record a video for heart rate estimation. These opportunistic moments define the opportunity when a user is holding the phone in his/her hand, facing up, and there are appropriate lighting conditions and minimal motion.

The systems and methods herein use the accelerometer data and proximity sensor to detect the position of the handheld device, and the systems and methods herein prune out cases (such as phone-in-pocket, phone-on-table etc.) by detecting if the phone is in the user's hand or not. For example, accelerometer and gyroscope data is used to detect the orientation and the rotation angle at which the phone is held in hand, which infers whether the phone's camera is facing toward the user's face or not. Hand motion (using the accelerometer) is also used for estimated readings, and the ambient light sensor is used to detect whether there is an appropriate illumination or not.

Video-based heart and respiration rate tracking processes often use a minimum amount of video (e.g., at least 15 seconds), and therefore, the systems and methods herein combine sensory information with soft-sensory features (i.e. backlight on/off, application in foreground, touch events) to train a decision-tree based classifier, in order to predict when heart rate tracking is possible.

Thus, the systems and methods herein provide frequent and regular (e.g., daily) heart rate monitoring to track the health of people, and this helps identify vulnerabilities to various health risks. Because the systems and methods herein are pervasive and non-obtrusive, they provide just-in-time health interventions and are a highly useful building block to provide effective prescriptions to the users.

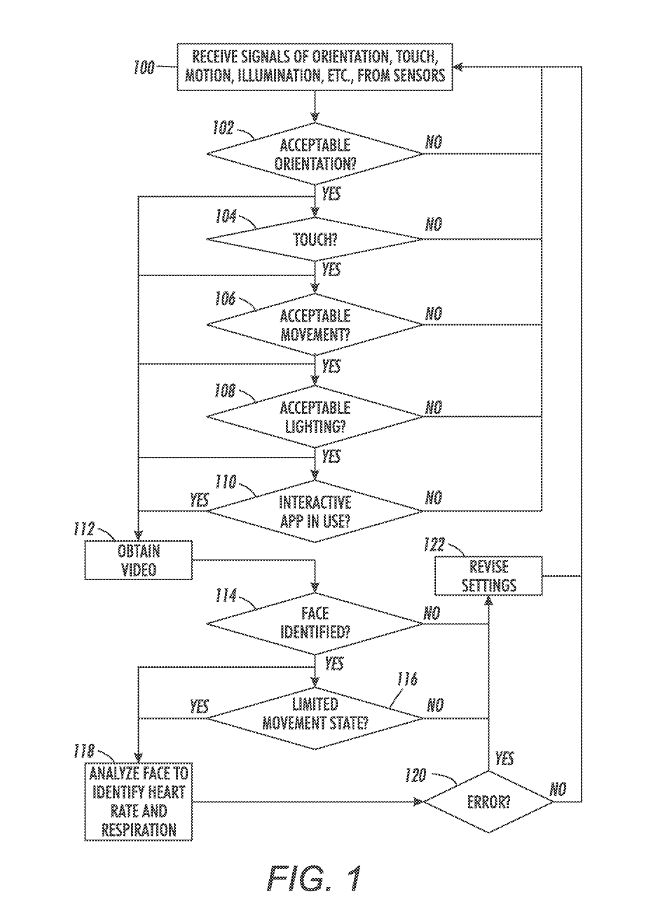

FIG. 1 is flowchart illustrating exemplary methods herein. In item 100, these methods are shown to receive (e.g., by a processor of a handheld apparatus) orientation signals from an orientation sensor of the handheld apparatus. For example, this “orientation sensor” can be one or more devices such as a gyroscope, an accelerometer, a proximity sensor, a light sensor, etc.

For example, the orientation signals indicate the direction that a camera of the handheld apparatus is aimed. Thus, in item 102, the processor determines whether the orientation signals indicate that the direction that the camera is aimed is within a previously established acceptable orientation range and conditions are proper for obtaining videos. Processing returns to item 100 to start the process again if the orientation is not acceptable; however, process proceeds if the orientation is acceptable in item 102.

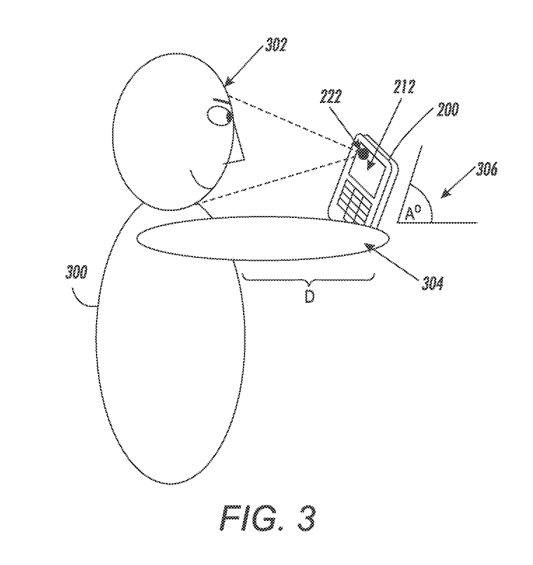

This “previously established acceptable orientation range” can be an angular range (e.g., relative to a gravitational source, such as the surface of the earth) having a sufficiently high likelihood of pointing to the face of the user (e.g., such as the camera pointing at 20°-80° relative to the ground). This is shown, for example, in FIG. 3 where the user 300 holds a handheld apparatus 200 in their hand 304 at an angle)(A° 306 such that the camera 222 of the handheld device 200 is oriented so as to capture video of the face 302 of the user 300. Note that in FIG. 3, the camera 222 is in a fixed position immediately adjacent the graphic user interface screen 212 and that the camera lens is positioned parallel to the graphic user interface screen 212 (causing the camera to be aimed in a direction perpendicular to the surface of the graphic user interface screen 212) and, therefore, the camera 222 is aimed (positioned) in a fixed position to capture video of items (e.g., faces) that are positioned perpendicular to the surface of the graphic user interface screen 212. Therefore, this previously established acceptable orientation range)(A° should have a likelihood of pointing the camera 222 from the hand of the user 304 to the face of the user 302.

Also, in item 100, such methods can also receive, by the processor, touch signals from a touch sensor of the handheld apparatus. The touch signals indicate whether the handheld apparatus is contacting the user. Thus, in item 104, systems and methods herein use the processor to determine whether the user is touching the handheld apparatus. With touch signals that can indicate the handheld device is within the user's hand 304, the acceptable orientation in item 102 can be narrowed (e.g., camera pointing at 35°-65° relative to the ground) to allow systems and methods herein to more accurately determine when to obtain video from the camera. Therefore, the process of determining whether the orientation of the camera is acceptable in item 102 can include input not only from gyroscopes, accelerometers, proximity sensors, cameras, etc., but also from touch sensors, etc.

Additionally, in item 100, such methods can receive motion signals from the accelerometer that indicate the amount of movement of the camera. Therefore, as shown in item 106, these methods can be limited to automatically obtaining video only when the amount of movement of the camera is below a previously established movement limit based on the movement signals from the accelerometer. This previously established movement limit can be any unit of force or speed measurement (G-force, pounds of force, meters per second, etc.). Further, the previously establish movement limit can be tailored to allow some movement, such as movement associated with a user interacting with an actual or virtual keyboard of the graphic user interface of the handheld apparatus. Indeed, in some examples, these systems and methods herein may require that there be a minimum amount of movement to ensure that the user is actually interacting with the handheld device, and that the handheld device is not resting on an inanimate surface far from the user's possession.

Similarly, in item 100, such methods can receive illumination signals from the light sensor indicating the illumination level of the environment surrounding the camera. Therefore, as shown in item 108, these methods can control the camera to automatically obtain video only when the illumination level is above a previously established illumination limit. Such processing prevents video from being captured when the handheld apparatus is in the user's pocket or when the user is in a low-illumination environment that would not allow the camera to obtain video of sufficient quality to perform heart rate and respiration analysis. The previously established illumination limit is dependent upon the lowlight capability of the camera and, therefore, different camera devices will have different previously established illumination limits.

As shown in item 110, video may only be obtained when an application involving extended user interaction with the graphic user interface of the handheld apparatus (e.g., texting, gaming, video conferencing, etc.) is actively being used by the user of the handheld apparatus. Therefore, in item 110, the methods herein use the processor to determine which functions (programs, applications, etc.) of the handheld apparatus are currently active; and of those functions that are currently active, the processor also determines which functions the user is currently interacting with.

Thus, in item 110, the methods herein also look to see whether the functions currently being interacted with by the user are those that require the user to observe (look at) the graphic user interface (e.g., display screen) of the handheld apparatus. Systems and methods herein increase the probability that video captured by the camera will include the user's face within the field of view of the camera if the user is currently (actively) interacting with an application or program that requires the user to look at the graphic user interface of the handheld apparatus (where the camera utilized to obtain the video is adjacent the graphic user interface, and is aimed perpendicular to the flat surface of the graphic user interface screen that the user looks at when interacting with the application or program). Programs that require the user to look at the graphic user interface can be previously identified, or categories of programs can be selected (e.g., texting programs, gaming programs, video conferencing programs, etc.) in order to identify which functions have a sufficient likelihood of causing video captured by the camera adjacent to graphic user interface screen to include the user's face.

In order to limit situations in which the camera of the handheld device obtains video to those situations that have a high likelihood of obtaining video of the user's face (and thereby reduce battery consumption, save processing resources, save memory resources, increase processing speed of other activities, etc.) the methods and systems herein control the camera to automatically obtain video only when one or more conditions are present. For example, as shown in FIG. 1, the process of obtaining video in item 112, only occurs if one or more of decision boxes 102, 104, 106, 108, and/or 110 results in a positive (yes) determination.

Thus, as shown in FIG. 1, the processor controls the camera to automatically obtain video based on, for example, the orientation signals indicating that the direction that the camera is aimed is within a previously established acceptable orientation (102); and/or the touch signals indicating that the handheld apparatus is contacting the user in item (104); and/or the amount of movement of the camera is below a previously established movement limit (106); and/or the illumination level is above a previously established illumination limit (108); and/or the functions currently being interacted with by the user are those that require the user to look at the graphic user interface (110); etc.

In item 112, the video is obtained for at least a minimum amount of time to allow subsequent processing to identify a heart rate. For example, certain processing may require a minimum of 15 seconds of video in order to identify heart rate. Alternatively, video can be obtained for as long as one or more of conditions 102, 104, 106, 108, and/or 110 are present. In addition, other time amount maximums can be established in order to decrease memory utilization, decrease battery consumption, maintain privacy restrictions, etc. Thus, in a non-limiting example, video may only be saved for subsequent processing in item 112 if one or more of conditions 102, 104, 106, 108, and/or 110 are present for at least 15 seconds. In other example, video may be obtained in item 112 so long as one or more of conditions 102, 104, 106, 108, and/or 110 are present for a minimum of 15 seconds and for a maximum of 5 minutes. Those ordinarily skilled in the art would understand that these time minimums and maximums are arbitrary, and that other time minimums and maximums could be utilized based upon the needs of the analysis discussed below.

In item 114, the systems and methods herein determine whether a face is present in the video obtained in item 112. In order to save processing resources, if a face is not identified, the flow proceeds to item 122 to potentially revise the settings within items 102, 104, 106, 108, and/or 110 in a learning process that constantly fine tunes such settings; and eventually returns to item 100 to start the process again; however, processing proceeds if a face is identified in item 114. The processing flow can proceed directly from item 114 to item 118 to immediately analyze the face to detect heart rate, respiration rate, emotional state, stress level, etc.; or, in the alternative, processing can flow from item 114 to item 116 to determine whether there is a limited movement state.

Facial recognition techniques are commonly known and the systems and methods herein are useful with all such techniques, whether currently known or developed in the future. Some exemplary face recognition techniques identify the presence of a face based upon shape and relative positions of the eyes, nose, and mouth. In addition, such face recognition techniques can rely upon skin tone signatures to help identify a face within video field of view, and such processes can be used in item 114.

Additionally, if the touch signal is received from a touch sensor in item 100, the face identification process in item 114 can be more restrictive and only identify faces that are within a specific size range. The size range can be determined, for example, based upon the field of view of the camera of the handheld apparatus used in conjunction with a known average distance that users tend to position the camera from their face when they are holding the handheld apparatus in their hand (e.g., distance D in FIG. 3). Therefore, if it is determined that the user is holding the handheld apparatus, processing herein can be restricted such that only faces that are within a specific size range (relative to the field of view of the camera) may be analyzed to determine heart rate and respiration in order to avoid using precious processing resources unnecessarily analyzing faces that are not the user's.

As shown in item 116, these methods can also determine whether the face in the video was captured in a limited movement state for more than the previously established minimum time amount (e.g., 15 second in the example above). In order to avoid using precious processing resources unnecessarily, if the video does not contain a limited movement state for at least one establish minimum time amount, the flow proceeds to item 122 to potentially revise the settings within items 102, 104, 106, 108, and/or 110 in a learning process that constantly fine tunes such settings; and eventually returns to item 100 to start the process again; however, if there is a limited movement state, processing proceeds to item 118 to analyze the face to detect heart rate, respiration rate, emotional state, stress level, etc.

For example, this “limited movement state” could be a state where the face identified in item 114 stays within a certain location of the video field of view during the previously established amount of time, such as an initial location (e.g., where this “initial location” is where the face was initially located within the video field of view when the video capture started, or where the face stays within a percentage (e.g., 90%, 80%, etc.) of that initial location, etc.). Thus, for example, a face boundary may be established relative to the video field of view in item 116 at some point after of the face is identified in item 114. Then, item 116 makes a determination that there is a limited movement state if the face does not depart from the face boundary buy more then a predetermined percentage (e.g., 10%, 20%, etc.) during the entire established minimum time amount.

As noted above, the orientation sensor can include an accelerometer, and force and motion signals can be received in item 100 from the accelerometer that indicate the amount of force and movement experienced by the camera while the video was obtained in item 112. If these motion signals are available, processing in item 116 may only determine that there is a limited movement state if the amount of movement of the camera while the video was obtained (as determined by the force and motion signals) is below a previously established movement limit (e.g., based on force (G) or distance movement).

Therefore, the processing in item 116 may look not only to determine whether a face is maintained within the initial location of the video field of view, but such processing can also determine whether any strong force or sharp movement was experienced by the handheld apparatus during the time when the video was obtained. Such processes again avoid using precious processing resources unnecessarily by preventing video that is potentially blurry (as a result of various shocks that may be received by the hand held apparatus) from being subjected to the heart rate and respiration analysis.

When processing proceeds to item 118, these methods automatically analyze changes of the face within the video over time to identify the heart rate, respiration rate, emotional state, stress level, etc., exhibited by the face within the video using the processor. For example, in item 118 the analyzing process can identify volumetric changes in blood vessels visible on the face, based upon fluctuations in amount of ambient light reflected from the blood vessels. More specifically, by capturing a sequence of images of red, green and blue (RGB), color sensors pick up a mixture of reflected signals, where each color sensor records a mixture of the original source signals with slightly different weights. During a cardiac cycle, volumetric changes in the blood vessels modify the path length of the incident ambient light, which in turn changes the amount of light reflected and detected by the RGB sensors, and this information is used to determine the heart rate and similar measures can determine respiration. Therefore, by analyzing the pixel-to-pixel changes that occur between different frames of the video, the systems and methods herein can determine many measurements, such as heart rate, respiration, etc. Similarly, the user's emotional state, stress level, etc., can be determined by interpreting facial gestures, eye movements, eye blink rates, etc.; and such measurements can be used alone and or in combination with the identified heart rate and respiration rate in order to infer emotional state, stress level, etc.

After identifying the heart rate, respiration rate, emotional state, stress level, etc. in item 118, processing analyzes the heart rate, respiration rate, emotional state, stress level, etc. identified in item 118. If any item is outside minimum or maximum limits, item 120 determines that the heart rate, respiration rate, emotional state, stress level, etc., contains an error, and processing proceeds to item 122 to potentially revise the settings within items 102, 104, 106, 108, and/or 110 in a learning process that constantly fine tunes such settings. Otherwise, if the heart rate, respiration rate, emotional state, stress level, etc. are within minimum or maximum limits, processing again returns to item 100 to repeat such processing and identifies the heart rate, respiration rate, emotional state, stress level, etc. at a later time. Such minimum and maximum limits utilized in item 120 can be fixed limits (e.g. heart rate maximum: 200; heart rate minimum: 40; respiration rate maximum 50; respiration rate minimum 5; etc.) or can be percentage limits from a user's historically maintained rates (e.g., within 20%, 30%, 40%, etc., of the user's historically maintained minimums for maximums).

If resources of the computerized device were utilized to obtain video in item 112, but a face was not identified in item 114, or there was not a limited movement state in item 116, processing proceeds to item 122 to revise the settings to prevent unnecessarily utilizing such video recording resources for situations where a face will not be identified or where a limited movement state does not exist. Similarly, if the heart rate, respiration rate, emotional state, stress level, etc., identified in item 118 is determined erroneous in item 120, the settings again can be revised in item 122 to prevent unnecessarily utilizing computing resources to identify an erroneous heart rate, respiration rate, emotional state, stress level, etc.

More specifically, the process of revising the settings in item 122 is a learning process that uses a decision tree based classifier to analyze the orientation data, touch data, movement data, lighting data, interactive application use data, etc., that were found acceptable in items 102, 104, 106, 108, and/or 110 to identify specific orientations, touch conditions, movement conditions, lighting conditions, interactive application used conditions that regularly results in a face not being identified in item 114, a limited movement state not being found an item 116, and/or erroneous results being calculated in item 118. Therefore, the learning process in item 122 revises specific settings within the evaluation processes performed in items 102, 104, 106, 108, and/or 110 (based on failures within items 114, 116 and/or 118) to continuously revise and fine tune the settings within items 102, 104, 106, 108, and/or 110 to only utilize video capture resources in item 112 and analysis resources and item 118 for situations which are highly likely to produce heart rates, respiration rates, emotional states, stress levels, etc., that are error-free.

For example, the learning engine in item 122 learns user-specific as well as aggregated rules, and stores previous instances and learns from them. In one example, the learning process in item 122 can determine that whenever the user uses gaming applications, there is too much motion, and when gaming applications are being interacted with, it is not a good time to record a video feed. In another example, item 122 can determine that whenever the user uses a video-based application (such as video conferencing applications, etc.) the motion is small and it is a good time to track the heart rate. Again, such rules are learned using a decision tree based classifier from previous instances and this knowledge is used for making future decisions in items 102, 104, 106, 108, 110, 114, 116, etc.

Processing from item 118 can be repeated by starting again in item 100 according to any schedule such as, every 15 minutes, hourly, twice a day, daily, weekly, etc.

The hardware described herein plays a significant part in permitting the foregoing methods to be performed, rather than function solely as a mechanism for permitting a solution to be achieved more quickly, (i.e., through the utilization of a computer for performing calculations). As would be understood by one ordinarily skilled in the art, the processes described herein cannot be performed by human alone and instead such processes can only be performed by a machine because evaluating electronic signals generated by orientation sensors, gyroscopes, touch sensors, light sensors, etc., are not activities that are capable of being performed by human. Similarly, the process of determining a user's heart rate based upon observing subtle pixel-by-pixel differences between different video frames is an activity that can only be performed by machines and cannot be performed by humans. Additionally, processes such as evaluating electronic signals output by various sensors and evaluating pixels within video frames are fundamental features of the systems and methods described herein and are not merely post-solution activity, because these systems and methods decide whether to obtain video based upon the electronic signals output by the various sensors, and these systems and methods determine the heart rate and respiration rate by analyzing the pixels within the video frames.

Additionally, the methods herein solve many highly complex technological problems. For example, as mentioned above, wearable devices that monitor heart or respiration rates are obtrusive and users often forget to wear them. Other non-wearable products require the users to manually start a heart rate tracking application periodically, or to take action in response to periodic notifications. However, users often miss notifications and forget to periodically use such applications and devices in the real world. Systems and methods herein solve these technological problems by providing devices that are pervasive and non-obtrusive, which results in tracking health statistics on a very regular basis. In addition, by only capturing video and processing such video when specific conditions are present, the systems and methods herein reduce the amount of processing resources and electronic storage that a device must contain, which reduces the cost and size (and increases the speed) of the handheld device. By granting such benefits, the methods herein reduce the amount and complexity of hardware and software needed to be utilized, thereby solving a substantial technological problem experience today.

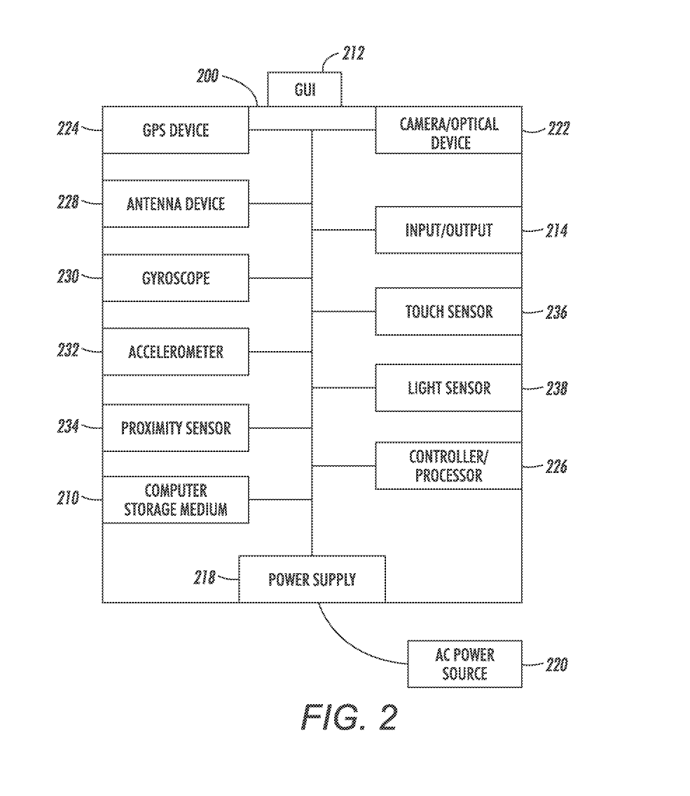

FIG. 2 illustrates a computerized device 200, which can be used with systems and methods herein and can comprise, for example, a print server, a personal computer, a portable computing device, etc. The computerized device 200 includes a controller/tangible processor 226 and a communications port (input/output) 214 operatively connected to the tangible processor 226 and to the computerized network external to the computerized device 200. Also, the computerized device 200 can include at least one accessory functional component, such as a graphical user interface (GUI) assembly 212. The user may receive messages, instructions, and menu options from, and enter instructions through, the graphical user interface or control panel 212.

The input/output device 214 is used for communications to and from the computerized device 200 and comprises a wired device or wireless device (of any form, whether currently known or developed in the future). The tangible processor 226 controls the various actions of the computerized device. A non-transitory, tangible, computer storage medium device 210 (which can be optical, magnetic, capacitor based, etc., and is different from a transitory signal) is readable by the tangible processor 226 and stores instructions that the tangible processor 226 executes to allow the computerized device to perform its various functions, such as those described herein. Thus, as shown in FIG. 2, a body housing has one or more functional components that operate on power supplied from an alternating current (AC) source 220 by the power supply 218. The power supply 218 can comprise a common power conversion unit, power storage element (e.g., a battery, etc), etc.

FIG. 2 also illustrates that the computerized device 200, is a special-use device such as a smartphone, tablet, or other special-purpose portable computerized element that is easily carried by a user. Such devices are special-purpose devices distinguished from general-purpose computers because such devices include specialized hardware, such as: specialized processors (e.g., containing specialized filters, buffers, application specific integrated circuits (ASICs), ports, etc.) that are specialized for phone communications, for use with cellular networks, etc.; specialized graphic user interfaces 212 (that are specialized for reduced power consumption, reduced size, antiglare, etc.); antenna 228 (that are specialized for phone communications, for use with cellular networks, etc.); specialized converters; GPS equipment 224; cameras and optical devices 222 (that are specialized for obtaining images with camera components, have specialized batteries, have specialized protective cases for use in harsh environments, etc.).

Thus, as shown above, exemplary handheld apparatuses herein include, among other components, a processor 226, a camera 222 operatively (meaning directly or indirectly) connected to the processor 226, an orientation sensor (which can be one or more devices such as a gyroscope 230, an accelerometer 232, a proximity sensor 234 etc.) operatively connected to the processor 226, a touch sensor 236 and a light sensor 238 operatively connected to the processor 226, etc. The processor 226 receives, for example, orientation signals from the orientation sensor(s) indicating the direction that the camera 222 is aimed, touch signals from the touch sensor 236 indicating the handheld apparatus is contacting the user (e.g., is being held in the user's hand), etc.

The processor 226 controls the camera 222 to automatically obtain video based on, for example, the orientation signals indicating the direction that the camera 222 is aimed is within a previously established acceptable orientation range (e.g., at an angle toward the user's face) and/or the touch signals indicating the handheld apparatus is contacting the user. Thus, in one example, the previously established acceptable orientation range can be an angular range (e.g., relative to a gravitational source, such as the surface of the earth) having a sufficiently high likelihood of pointing from the hand of the user to the face of the user (e.g., such as the camera 222 pointing at 30°-60° relative to the ground).

Additionally, the processor 226 can receive motion signals from the accelerometer 232 that indicate the amount of movement of the camera 222; and the processor 226 can be limited to controlling the camera to automatically obtain video only when the amount of movement of the camera 222 is below a previously established movement limit. Similarly, the processor 226 can receive illumination signals from the light sensor 238 indicating the illumination level of the environment surrounding the camera 222; and the processor 226 can control the camera 222 to automatically obtain video only when the illumination level is above a previously established illumination limit. Also, the processor 226 can control the camera 222 to automatically obtain video only when an application involving extended user interaction with the graphic user interface 212 of the handheld apparatus 200 is actively being used by the user of the handheld apparatus.

The processor 226 automatically recognizes items within the video to identify the user's face within and the video. The processor 226 can then automatically analyze changes of the face within the video over time to identify the heart rate, respiration rate, emotional state, stress level, etc., exhibited by the face within the video. More specifically, the processor 226 can analyze changes of the face by identifying volumetric changes in blood vessels visible on the face (e.g., based upon fluctuations in amount of ambient light reflected from such blood vessels), etc.

While some exemplary structures are illustrated in the attached drawings, those ordinarily skilled in the art would understand that the drawings are simplified schematic illustrations and that the claims presented below encompass many more features that are not illustrated (or potentially many less) but that are commonly utilized with such devices and systems. Therefore, Applicants do not intend for the claims presented below to be limited by the attached drawings, but instead the attached drawings are merely provided to illustrate a few ways in which the claimed features can be implemented.

Many computerized devices are discussed above. Computerized devices that include chip-based central processing units (CPU's), input/output devices (including graphic user interfaces (GUI), memories, comparators, tangible processors, etc.) are well-known and readily available devices produced by manufacturers such as Dell Computers, Round Rock Tex., USA and Apple Computer Co., Cupertino Calif., USA. Such computerized devices commonly include input/output devices, power supplies, tangible processors, electronic storage memories, wiring, etc., the details of which are omitted herefrom to allow the reader to focus on the salient aspects of the systems and methods described herein. Similarly, printers, copiers, scanners and other similar peripheral equipment are available from Xerox Corporation, Norwalk, Conn., USA and the details of such devices are not discussed herein for purposes of brevity and reader focus.

A “pixel” refers to the smallest segment into which an image can be divided. Received pixels of an input image are associated with a color value defined in terms of a color space, such as color, intensity, lightness, brightness, or some mathematical transformation thereof. Pixel color values may be converted to a chrominance-luminance space using, for instance, a RBG-to-YCbCr converter to obtain luminance (Y) and chrominance (Cb,Cr) values. It should be appreciated that pixels may be represented by values other than RGB or YCbCr.

Thus, an image input device is any device capable of obtaining color pixel values from a color image. The set of image input devices is intended to encompass a wide variety of devices such as, for example, digital document devices, computer systems, memory and storage devices, networked platforms such as servers and client devices which can obtain pixel values from a source device, and image capture devices. The set of image capture devices includes, cameras, photography equipment. Modern image capture devices typically incorporate a charge-coupled device (CCD). The image capture devices produce a signal of the image data. Such a digital signal contains information about pixels such as color value, intensity, and their location within the image.

In addition, terms such as “right”, “left”, “vertical”, “horizontal”, “top”, “bottom”, “upper”, “lower”, “under”, “below”, “underlying”, “over”, “overlying”, “parallel”, “perpendicular”, etc., used herein are understood to be relative locations as they are oriented and illustrated in the drawings (unless otherwise indicated). Terms such as “touching”, “on”, “in direct contact”, “abutting”, “directly adjacent to”, etc., mean that at least one element physically contacts another element (without other elements separating the described elements). Further, the terms automated or automatically mean that once a process is started (by a machine or a user), one or more machines perform the process without further input from any user. In the drawings herein, the same identification numeral identifies the same or similar item.

It will be appreciated that the above-disclosed and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims. Unless specifically defined in a specific claim itself, steps or components of the systems and methods herein cannot be implied or imported from any above example as limitations to any particular order, number, position, size, shape, angle, color, or material.

What is claimed is:

1. A handheld apparatus comprising:
a processor;
a camera operatively connected to said processor; and
an orientation sensor operatively connected to said processor,
said processor receiving orientation signals from said orientation sensor indicating a direction that said camera is aimed,
said processor controlling said camera to automatically obtain video based on said orientation signals indicating said direction that said camera is aimed being within a previously established acceptable orientation range,
said processor automatically recognizing items within said video to identify a face within and said video, and
said processor automatically analyzing changes of said face within said video over time to identify at least one of a heart rate and a respiration rate exhibited by said face within said video.

2. The handheld apparatus according to claim 1, further comprising a light sensor,
said processor receiving illumination signals from said light sensor indicating an illumination level of an environment surrounding said camera, and
said processor controlling said camera to automatically obtain video only when said illumination level is above a previously established illumination limit.

3. The handheld apparatus according to claim 1, said orientation sensor comprising an accelerometer,
said processor receiving motion signals from said accelerometer indicating an amount of movement of said camera, and
said processor controlling said camera to automatically obtain video only when said amount of movement of said camera is below a previously established movement limit.

4. The handheld apparatus according to claim 1, said previously established acceptable orientation range comprising an angular range, relative to a gravitational source, having a likelihood of pointing from a hand of a user to said face of said user.

5. The handheld apparatus according to claim 1, said processor controlling said camera to automatically obtain video only when an application involving extended user interaction with said handheld apparatus is actively being used by a user of said handheld apparatus.

6. A handheld apparatus comprising:
a processor;
a camera operatively connected to said processor;
an orientation sensor operatively connected to said processor; and
a touch sensor operatively connected to said processor,
said processor receiving orientation signals from said orientation sensor indicating a direction that said camera is aimed,
said processor receiving touch signals from said touch sensor indicating said handheld apparatus contacting a user,
said processor controlling said camera to automatically obtain video based on said touch signals indicating said handheld apparatus contacting said user and said orientation signals indicating said direction that said camera is aimed being within a previously established acceptable orientation range,
said processor automatically recognizing items within said video to identify a face within and said video, and
said processor automatically analyzing changes of said face within said video over time to identify at least one of a heart rate, a respiration rate, an emotional state, and a stress level exhibited by said face within said video.

7. The handheld apparatus according to claim 6, further comprising a light sensor,
said processor receiving illumination signals from said light sensor indicating an illumination level of an environment surrounding said camera, and
said processor controlling said camera to automatically obtain video only when said illumination level is above a previously established illumination limit.

8. The handheld apparatus according to claim 6, said orientation sensor comprising an accelerometer,
said processor receiving motion signals from said accelerometer indicating an amount of movement of said camera, and
said processor controlling said camera to automatically obtain video only when said amount of movement of said camera is below a previously established movement limit.

9. The handheld apparatus according to claim 6, said previously established acceptable orientation range comprising an angular range, relative to a gravitational source, having a likelihood of pointing from a hand of a user to said face of said user.

10. The handheld apparatus according to claim 6, said processor controlling said camera to automatically obtain video only when an application involving extended user interaction with said handheld apparatus is actively being used by a user of said handheld apparatus.

11. A method comprising:
receiving, by a processor of a handheld apparatus, orientation signals from an orientation sensor of said handheld apparatus, said orientation signals indicating a direction that a camera of said handheld apparatus is aimed;
controlling said camera to automatically obtain video based on said orientation signals indicating said direction that said camera is aimed being within a previously established acceptable orientation range using said processor;
automatically recognizing items within said video to identify a face within said video using said processor; and
automatically analyzing changes of said face within said video over time to identify at least one of a heart rate and a respiration rate exhibited by said face within said video using said processor.

12. The method according to claim 11, said handheld apparatus further comprising a light sensor, said method further comprising:
receiving, by said processor, illumination signals from said light sensor indicating an illumination level of an environment surrounding said camera, and
controlling, by said processor, said camera to automatically obtain video only when said illumination level is above a previously established illumination limit.

13. The method according to claim 11, said orientation sensor comprising an accelerometer, said method further comprising:
receiving, by said processor, motion signals from said accelerometer indicating an amount of movement of said camera, and
controlling, by said processor, said camera to automatically obtain video only when said amount of movement of said camera is below a previously established movement limit.

14. The method according to claim 11, said previously established acceptable orientation range comprising an angular range, relative to a gravitational source, having a likelihood of pointing from a hand of a user to said face of said user.

15. The method according to claim 11, said controlling said camera automatically obtaining said video only when an application involving extended user interaction with said handheld apparatus is actively being used by a user of said handheld apparatus.

16. A method comprising:
receiving, by a processor of a handheld apparatus, orientation signals from an orientation sensor of said handheld apparatus, said orientation signals indicating a direction that a camera of said handheld apparatus is aimed;
receiving, by said processor, touch signals from a touch sensor of said handheld apparatus, said touch signals indicating said handheld apparatus contacting a user;
controlling said camera to automatically obtain video based on said touch signals indicating said handheld apparatus contacting said user and said orientation signals indicating said direction that said camera is aimed being within a previously established acceptable orientation range using said processor;
automatically recognizing items within said video to identify a face within said video using said processor; and
automatically analyzing changes of said face within said video over time to identify at least one of a heart rate, a respiration rate, an emotional state, and a stress level exhibited by said face within said video using said processor.

17. The method according to claim 16, said handheld apparatus further comprising a light sensor, said method further comprising:
receiving, by said processor, illumination signals from said light sensor indicating an illumination level of an environment surrounding said camera, and
controlling, by said processor, said camera to automatically obtain video only when said illumination level is above a previously established illumination limit.

18. The method according to claim 16, said orientation sensor comprising an accelerometer, said method further comprising:
receiving, by said processor, motion signals from said accelerometer indicating an amount of movement of said camera while said video was obtained; and
controlling said camera to automatically obtain video only when said amount of movement of said camera while said video was obtained is below a previously established movement limit, using said processor.

19. The method according to claim 16, said previously established acceptable orientation range comprising an angular range, relative to a gravitational source, having a likelihood of pointing from a hand of a user to said face of said user.

20. The method according to claim 16, said controlling said camera automatically obtaining said video only when an application involving extended user interaction with said handheld apparatus is actively being used by a user of said handheld apparatus.
